(19) United States
(12) Patent Application Publication
(10) Pub. No.: US 2009/0216197 A1
(43) Pub. Date: Aug. 27, 2009

(54) Urinary Catheter Holder

(75) Inventor: Ronald D. Russo, Barrington, RI (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Dale Medical Products, Inc.

(21) Appl. No.: 12/389,785

(22) Filed: Feb. 20, 2009

Related U.S. Application Data
(60) Provisional application No. 61/030,727, filed on Feb. 22, 2008.

(51) Int. Cl. A61M 25/02

(52) U.S. Cl. 604/179; 604/327

(57) ABSTRACT

A catheter holder is provided. The catheter holder comprises a cushioned platform for shielding a patent's skin from rigid portions of a catheter. The cushioned platforms comprises a central portion and two sections extending in opposite directions from the central portion, each section having a width that is less than a width of the central portion. The catheter holder further comprises a stretchable band extending from at least one of the two sections and a catheter locking mechanism disposed on the central portion of the cushioned platform, the locking mechanism being secured to the central portion at a first location and at least one tab for preventing rotational motion of the rigid portions of the catheter.

This application claims priority under 35 U.S.C. § 119 to U.S. Provisional Patent Application No. 61/030,727, filed Feb. 22, 2008, which is herein incorporated by reference in its entirety.

### FIELD

The present disclosure relates to the field of medical device holders, and more specifically, to urinary catheter holders.

### BACKGROUND

Various tube holders are used to secure medical tubes such as urinary catheters (e.g., Foley catheters). Some urinary catheter holders are designed to secure a catheter to a leg of a patient, thereby preventing unintended movement, which can be uncomfortable. Such catheter holders typically are designed to secure the proximal end of the catheter to a patient's thigh. The proximal end of the catheter can include a Y-port. One such catheter holder is described in U.S. Pat. Nos. 5,941,896 and 4,445,894, both issued to Kovacs and incorporated herein by reference in their entirety.

The catheter Y-port is connected to a urinary drainage system and generally includes long, coiled drainage tubing and one or more rigid, molded plastic parts, such as a urine sampling port. Once inserted into a patient, the uncoiled drainage tubing can rotate, causing internal catheter movement and irritation. Such irritation is a common cause of urinary tract infections. Previously, catheter urine sampling ports included a needle puncture port for syringe aspiration. However, more recently, urine sampling ports have needleless access system, and while needleless urine sampling ports can reduce the risk of needle stick injury to clinicians and patients, they often include a high profile with sharp corners and deep luer threads that can dig into a patient's skin. This may promote skin breakdown, irritation, and possible skin infection.

The present disclosure provides a urinary catheter holder with a built-in skin protective cushion and a locking mechanism for preventing rotational movement of the rigid molded part.

### SUMMARY

According to one exemplary embodiment, a catheter holder is provided. The catheter holder comprises a cushioned platform for shielding a patient's skin from rigid portions of a catheter. The cushioned platform comprises a central portion and two sections extending in opposite directions from the central portion, each section having a width that is less than a width of the central portion. The catheter holder further comprises a stretchable band extending from at least one of the two sections and a catheter locking mechanism disposed on the central portion of the cushioned platform, the locking mechanism being secured to the central portion at a first location and including at least one tab for preventing rotational motion of the rigid portions of the catheter.

According to another exemplary embodiment, a method for securing a catheter to the leg of a patient is provided. The method comprises positioning a cushioned platform for shielding a patient's skin from rigid portions of a catheter on a leg of a patient. The cushioned platform comprises a central portion and two sections extending in opposite directions from the central portion, each section having a width that is less than a width of the central portion. The method further comprises wrapping a stretchable band extending from one of the two sections of the cushioned platform around the leg of the patient and securing the stretchable band to a loop material located on the other of the two sections of the cushioned platform. The method also comprises positioning a portion of a catheter on a catheter locking mechanism disposed on the central portion of the cushioned platform and securing the catheter to the central portion using the locking mechanism.

According to another exemplary embodiment, a catheter and catheter holder assembly are provided. The assembly comprises a urinary catheter having a Y-port at one end and a cushioned platform for shielding a patient's skin from rigid portions of a catheter. The cushioned platform comprises a central portion and two sections extending in opposite directions from the central portion, each section having a width that is less than a width of the central portion. The assembly further comprises a stretchable band extending from at least one of the two sections and a catheter locking mechanism disposed on the central portion of the cushioned platform, the locking mechanism being secured to the central portion at a first location and at least one tab for preventing rotational motion of the rigid portions of the catheter.

### BRIEF DESCRIPTION OF THE DRAWINGS

The accompanying drawings, which are incorporated in and constitute a part of this specification, provide exemplary embodiments of the disclosure and, together with the description, serve to explain exemplary embodiments consistent with the principles of the present invention.

FIG. 1 is a perspective view of a urinary catheter holder, according to certain exemplary embodiments.

FIG. 2 is a partial cross-sectional view of a material that can be used to produce a cushioned platform of the disclosed urinary catheter holders, according to certain exemplary embodiments.

FIG. 3 is a perspective view of a urinary catheter holder along with a urinary drainage system prior to closure of the locking mechanism, according to certain exemplary embodiments.

FIG. 4 is a top view of a urinary catheter holder, according to certain exemplary embodiments.

FIG. 5 is a top view of a urinary catheter holder and catheter secured to a patient's limb after closure of the locking mechanism, according to certain exemplary embodiments.

FIG. 6 illustrates steps of a method for securing a urinary catheter to a patient's leg, according to certain exemplary embodiments.

### DETAILED DESCRIPTION

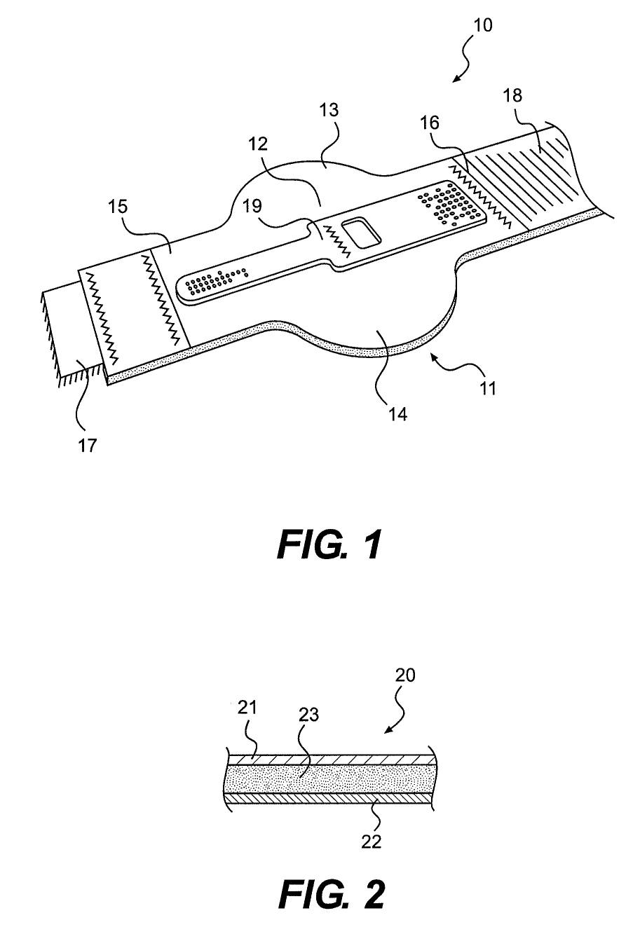

FIG. 1 is a perspective view of a urinary catheter holder 10, according to one exemplary embodiment. The holder 10 includes a cushioned platform 11 having a central portion 12. The cushioned platform 11 comprises two sections 15, 16 extending outwardly in opposite directions from the central portion 12. In one exemplary embodiment, the sections 15 and 16 are approximately two inches wide, but a range of sizes (e.g., widths between about one inch and about three inches) are contemplated. The central portion 12 also includes widened extensions 13, 14 extending outwardly from the central portion 12, such that the two sections 15, 16 each have widths that are less than the width of the central portion 12. In one exemplary embodiment, the central portion 12 is four inches in total width, which is wide enough to provide total cushioning against the rigid molded component of typical urinary drainage systems. However, the central portion can have a width ranging from between about three inches up to about six inches.

The cushioned platform 11 can include a variety of shapes. For example, in one exemplary embodiment, the widened sections 13, 14 form an oval shape with sections 15, 16 extending on opposite sides. However, the cushioned platform 11 can include other shapes (e.g., rectangular, circular, diamond, pyramid, or square), as long as the platform is shaped to protect the skin of a patient from a catheter secured by the holder 10. In one exemplary embodiment, the two widened extensions 13, 14 are symmetrical mirror images of each other such that the holder 10 is universally adaptable to either the left or right limb of a patient. Since the holder 10 is universally adaptable, it can also be used to shift the leg band from one thigh to the other thigh after some time (e.g., several days) of use, or for particular patient comfort.

Additional components of the holder 10 can be attached to and/or extend from the ends of the sections 15, 16. In some embodiments, one of the two sections 15, 16 can include a hook or loop type material (e.g., Velcro® material) attached to or extending from the section. For example, as shown in FIG. 1, the holder 10 includes a hook material 17 extending from one section 15, and the other section 16 is attached to a stretchable band 18 that can be wrapped around the limb of a patient to allow the holder 10 to be secured to the limb. As shown, the hook material 17 and stretchable band 18 are sewn to the respective sections 15, 16, but the hook material 17 and stretchable band 18 can be secured to the sections 15, 16 using a variety of different attachment mechanisms such as sonic welding.

As shown in FIG. 1, the holder 10 also includes a locking mechanism 19 disposed on the central portion 12. As described in more detail below, the locking mechanism 19 can be used to secure a catheter to the holder 10. In some embodiments, the locking mechanism 19 is approximately centrally located on the cushioned platform 11 such that when the proximal end of a catheter is attached to the holder 10, rigid components of the catheter will rest on the cushioned platform 11, and not on the skin of a patient.

The cushioned platform 12 can be configured to protect the patient's skin from components of a catheter attached to the holder 10. In some embodiments, the platform can include a cushioned material having sufficient thickness to protect the skin.

In certain embodiments, the platform can include layered and/or laminated materials. For example, FIG. 2 is a partial cross-sectional view of a material 20 that can be used to produce the cushioned platform 11. As shown, in one exemplary embodiment, the cushioned platform 11 includes at least two layers, and can include three laminated layers. One layer includes an outer or top layer 21 formed of loop type material on at least a portion of its surface. Further, the second layer 23 is a cushioned material. The second layer cushioned material can include, for example, an open-cell polyurethane foam.

In one exemplary embodiment, the cushioned platform 11 includes a third layer 22 comprising a moisture-resistant material. Moisture resistant materials can be partially or completely impermeable to water. Suitable moisture resistant-materials can include non-woven polyolefin fabrics. The third layer 21 is positioned on a bottom surface of the cushioned platform to contact the skin of a patient when the holder 10 is secured to a patient's limb. The layers 21, 22, 23 are laminated together to form a soft, flexible, absorbent and cushioned, yet strong platform 11. The material 20 forms a stable, non-stretchy platform 11 such that the locking mechanism 19 remains in a fixed position, thereby forming a stable mechanism for securing the catheter independent of the stretchable band.

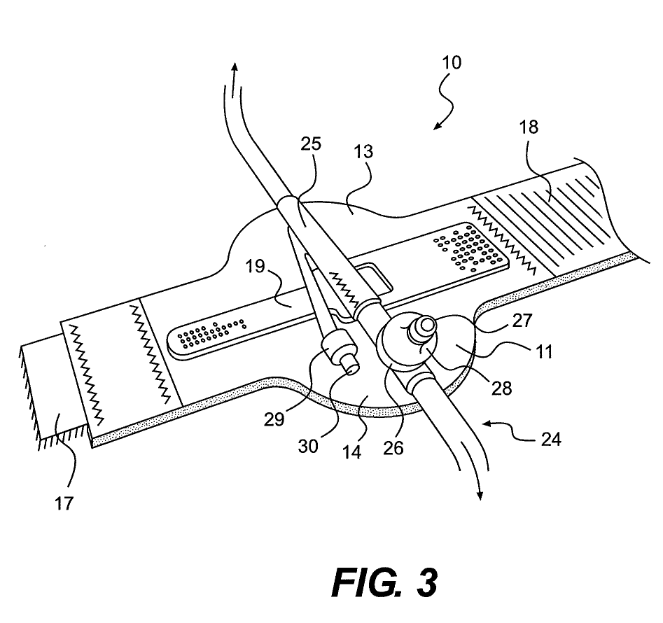

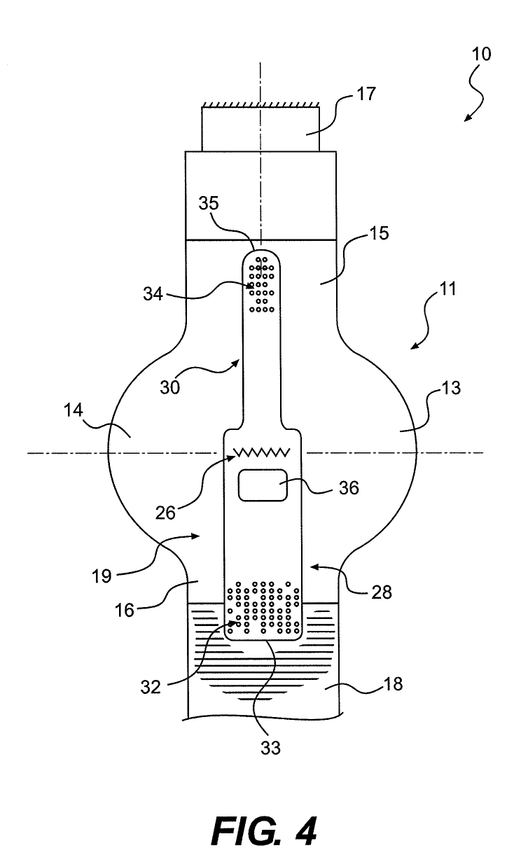

FIG. 3 is a perspective view of a urinary catheter holder 10 along with a urinary drainage system 24, according to certain embodiments. As shown, the drainage system 24 can include a pre-connected Foley catheter 25 having a Y-port formed from a connector 26 and balloon inflation port 29. The connector 26 can be formed of a rigid, molded material such as acrylonitrile butadiene styrene (ABS), polycarbonate, or polyvinylchloride (PVC) plastic. The connector 26 may include high profile built-in threaded luer lock needleless urine sampling port 27, typically also molded from the same plastic material. In some embodiments, the port 27 can include a luer-type connection with sharp threaded edges 28. In addition, the balloon inflation port 29 may also include a rigid, molded, sharp, plastic valve port 30.

FIG. 4 is a top view of a urinary catheter holder, according to one exemplary embodiment. As shown, the locking mechanism 19 is attached to the central portion 12 of the cushioned platform 11 at a first location 26, and the locking mechanism includes two tabs 28, 30 that can be used to attach a urinary drainage system 24 to the holder 10, as described in more detail below. In some embodiments, the tabs 28, 30 can include molded hook material sections 32, 34 positioned near ends 33, 35 of the tabs 28, 30, or the locking mechanism can be fabricated from die-cut Velcro® molded polypropylene hook material attached by sewing or sonic welding. In one exemplary embodiment, one of the tabs 28 includes an opening 36 sized such that the end 33 of the other tab 30 can be passed through the opening 36. While one style of locking mechanism is shown, many other types of catheter locking mechanisms can be utilized without departing from the broad scope of the disclosed urinary catheter holders.

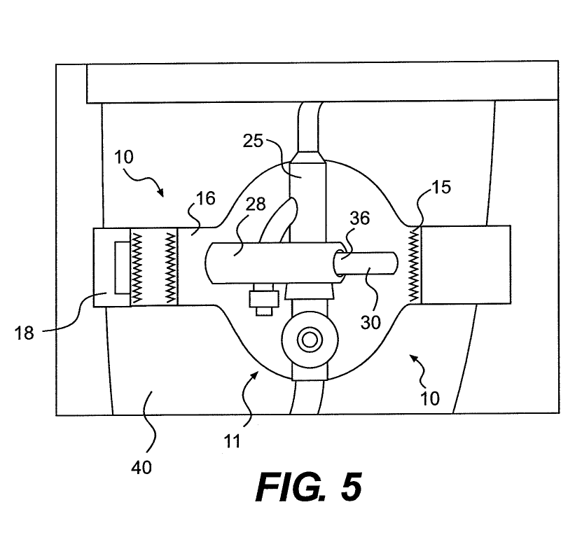

FIG. 5 is a top view of a urinary catheter holder and catheter secured to a patient's limb. As shown, the catheter 25 is secured to the cushioned platform 11 using the locking mechanism 19. As described more detail below, the catheter 25 is positioned on the platform 11, and one tab 30 is looped over the catheter 25 and passed through the opening 36 in the other tab 28. Next, the second tab 28 is looped over the catheter 25 in the opposite direction, and both tabs 28, 30 are secured to the surface of the platform using, for example, hook and loop materials. As such, locking mechanism 19 prevents rotational motion of the rigid molded portion of the catheter.

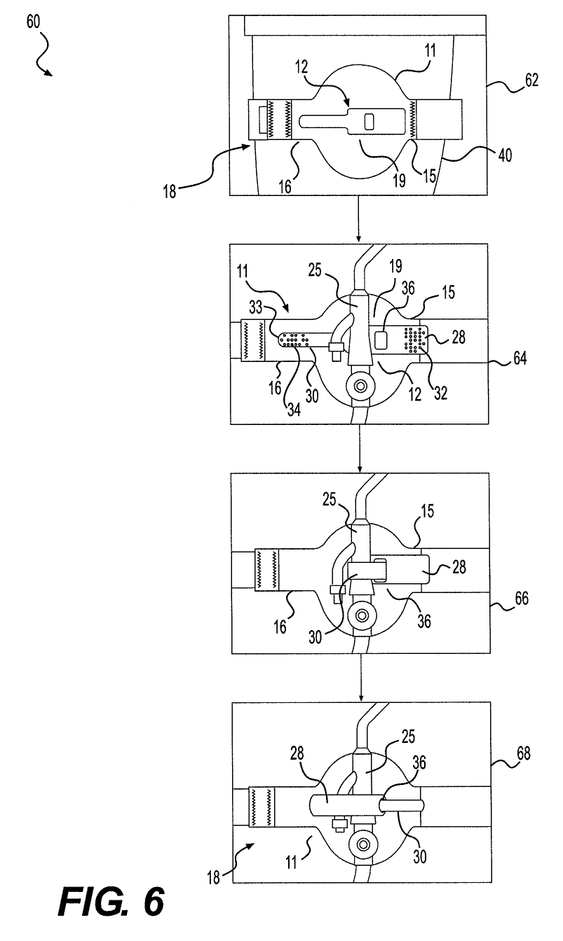

FIG. 6 illustrates steps of a method 60 for securing a urinary catheter to a patient's leg. As shown at step 62, a cushioned platform 11 of the holder 10 is first positioned on a patient's leg 40, the platform comprising a central portion 11 and two sections 15, 16 extending in opposite directions from the central portion 11. A stretchable band 18 extending from one of the sections 16 is wrapped around the patient's leg 40, and the band 18 is secured to a loop material section (not indicated in FIG. 6) on the other the section 15.

Next, as shown at step 64, a urinary catheter 25 is positioned on a catheter locking mechanism 19 disposed on the cushioned platform 11. As described above, the locking mechanism 19 is attached to the cushioned platform 11 and includes two tabs 28, 30. The end 33 of one tab 30 is then be passed over the catheter 25, as shown at step 66, and passed through an opening 36 in the other tab 28. Next, as shown at step 68, the other tab 28 is then looped over the catheter, and loop material sections 32, 34 located on the tabs 28, 30 are contacted with hook material on the surface of the cushioned platform 11 to secure the tabs 28, 30 and catheter 25 to the holder 10.

It will be apparent to those skilled in the art that additional various modifications and variations can be made consistent with the present disclosure. Other embodiments consistent with the tube holder will be apparent to those skilled in the art from consideration of the specification and practice of the disclosure. It is intended that the specification and examples be considered as exemplary only, with a true scope and spirit of the disclosure being indicated by the following claims.

1. A catheter holder, comprising:
a cushioned platform for shielding a patient's skin from rigid portions of a catheter, comprising:
a central portion; and
two sections extending in opposite directions from the central portion, each section having a width that is less than a width of the central portion;
a stretchable band extending from at least one of the two sections; and
a catheter locking mechanism disposed on the central portion of the cushioned platform, the locking mechanism being secured to the central portion at a first location and including at least one tab for preventing rotational motion of the rigid portions of the catheter.

2. The holder of claim 1, wherein the cushioned platform includes a layered and laminated material.

3. The holder of claim 2, wherein the cushioned platform includes three layers, comprising:
a top loop material layer;
a middle cushion layer; and
a bottom moisture resistant layer.

4. The holder of claim 1, wherein the cushioned platform includes a top surface including a loop type material on at least a portion of the top surface.

5. The holder of claim 4, wherein the locking mechanism includes a hook type material.

6. The holder of claim 5, wherein the locking mechanism includes two tabs extending from the first location.

7. The holder of claim 1, wherein one of the two sections extending from the central portion of the cushioned platform comprises a loop material.

8. A method for securing a catheter to the leg of a patient, comprising:
positioning a cushioned platform for shielding a patient's skin from rigid portions of a catheter on a leg of a patient, the cushioned platform comprising a central portion and two sections extending in opposite directions from the central portion, each section having a width that is less than a width of the central portion;
wrapping a stretchable band extending from one of the two sections of the cushioned platform around the leg of the patient;
securing the stretchable band to a loop material located on the other of the two sections of the cushioned platform;
positioning a portion of a catheter on a catheter locking mechanism disposed on the central portion of the cushioned platform; and
securing the catheter to the central portion using the locking mechanism.

9. The method of claim 8, wherein positioning the cushioned platform includes positioning a bottom moisture resistant layer of the cushioned platform against the skin of the patient and an outer loop material layer away from the skin of the patient.

10. The method of claim 8, wherein positioning the cushioned platform includes positioning a top surface including a loop type material away from skin of the patient.

11. The method of claim 10, wherein securing the catheter to the central portion using the locking mechanism comprises passing a first tab extending in a first direction from the catheter of the locking mechanism over the catheter and through an opening in a second tab extending in a second direction from the catheter; and
securing the first and second tabs to a top surface of the cushioned platform.

12. The method of claim 11, wherein the first and second tabs are secured to the top surface with loop material located on the first and second tabs.

13. A catheter and catheter holder assembly, comprising:
a urinary catheter having at least one rigid port;
a cushioned platform for shielding a patient's skin from rigid portions of a catheter, comprising:
a central portion; and
two sections extending in opposite directions from the central portion and each section having a width that is less than a width of the central portion;
a stretchable band extending from at least one of the two sections; and
a catheter locking mechanism disposed on the central portion of the cushioned platform, the locking mechanism being secured to the central portion at a first location and at least one tab for preventing rotational motion of the rigid portions of the catheter.

14. The assembly of claim 13, wherein the cushioned platform includes a layered and laminated material.

15. The assembly of claim 14, wherein the cushioned platform includes three layers, comprising:
a top loop material layer;
a middle cushion layer; and
a bottom moisture resistant layer.

16. The assembly of claim 13, wherein the at least one tab of the locking mechanism includes a hook type material.

17. The assembly of claim 16, wherein the locking mechanism includes two tabs and one of the tabs includes an opening sized to allow an end portion of the other tab to pass through the opening.

18. The assembly of claim 13, wherein the catheter includes a needleless sampling port.

19. A urinary catheter holder for securing a urinary drainage catheter and drainage system wherein the catheter and drainage system comprises a Y-port and a drainage connector, the holder comprising:
a cushioned platform providing a cushioned barrier underneath the drainage connector on the catheter, the platform having a central portion and two sections extending in opposite directions from the central portion, each section having a width that is less than the width of the central portion;
a stretchable leg band for securing the holder to a patient's limb; and
a catheter securement mechanism fastened on top of the platform for engagement with the Y-port on the catheter.

20. The holder of claim 19 wherein the central portion is between three to six inches in total width.
